(19) United States
(12) Patent Application Publication
(10) Pub. No.: US 2004/0115126 A1
(43) Pub. Date: Jun. 17, 2004

(54) Compositions and methods for the non-invasive detection of polypeptides in vivo

(75) Inventors: Brian D. Ross, Ann Arbor, MI (US); Alnawaz Rehemtulla, Plymouth, MI (US)

Correspondence Address: David A. Casimir, MEDLEN & CARROLL, LLP, Suite 350, 101 Howard Street, San Francisco, CA, 94105, US

(73) Assignee: The Regents of the University of Michigan, Ann Arbor, MI

(21) Appl. No.: 10/654,532

(22) Filed: Sep. 3, 2003

Related U.S. Application Data
(60) Provisional application No. 60/408,474, filed on Sep. 3, 2002.

(51) Int. Cl. A61K 51/00; C12Q 1/68; G01N 33/567; G01N 33/53; A61B 5/055

(52) U.S. Cl. 424/1.41; 424/9.34; 435/6; 435/7.2; 424/9.6

(57) ABSTRACT

Methods for the in vivo monitoring and measuring of proteins by constructing them as chimeric polypeptides are disclosed. Through the use of the chimeric polypeptides, such methods can be used to screen and identify compounds and events that affect the presence or absence of the proteins in the cell.

The present disclosure claims priority to U.S. Provisional Application No. 60/408,474, filed Sep. 3, 2002, the contents of which are hereby incorporated by reference.

### TECHNICAL FIELD

This invention relates to the fields of medicine and medical research.

### BACKGROUND

The ubiquitin-proteolysis (proteosome) pathway plays a fundamental role in selective protein degradation in cells. This pathway is involved in diverse cellular functions, such as cell cycle control, metabolic regulation, and signal transduction (Ciechanover (1994) Cell 79: 13-21; Yamao (1999) J. Eurochem. 125: 223-229). In general, the ubiquitin conjugation to the substrate proteins to be degraded is governed by three enzymatic steps. In the initial step, ubiquitin-activating enzyme (E 1) activates the ubiquitin by an ATP hydrolysis to form a thioester bond between the C-terminus of ubiquitin and a cysteine residue of the same E 1 enzyme. Then a transthiolation leads to the transfer of ubiquitin from E 1 to the second enzyme, ubiquitinconjugating enzyme (UBC or E2). Finally, ubiquitin is covalently ligated to the Lys residues of the substrate protein via the isopeptide bond by CTBC enzyme alone or together with a third enzyme, ubiquitin-protein ligase (E3). The 2 bS proteosome then recognizes and degrades the polyubiquitinated proteins.

Throughout the life of a normal cell, proteins are synthesized and degraded, such as by the ubiquitin-proteosome pathway as described above. A breakdown in this normal cycle may be indicative of a disease state or the potential of developing a diseased state. For example, inactivation of p53 is one of the most frequent molecular events in neoplastic transformation. Approximately 60% of all human tumors have mutations in both p53 alleles.

Wild-type p53 activity is regulated in large part by the proteosome-dependent degradation of p53, resulting in a short p53 half-life in unstressed and untransformed cells. Activation of p53 by a variety of stimuli, including DNA damage induced by genotoxic drugs or radiation, is accomplished by stabilization of wild-type p53, The stabilized and active p53 can result in either cell-cycle arrest or apoptosis. Surprisingly, the majority of tumor-associated, inactivating p53 mutations also result in p53 accumulation. Thus, constitutive elevation of p53 levels in cells is a reliable measure of p53 inactivation, whereas transiently increased p53 levels reflect a recent genotoxic stress.

The p53 gene product plays an important role in tumor suppression. This is best demonstrated by the fact that 60% of all human cancers carry a mutation in the p53 gene. In addition, patients having Li-Fraumeni syndrome, due to inheritance of a defective p53 gene, are much more prone to develop cancers. Similarly, genetically engineered mice lacking the p53 gene are also more prone to develop cancer. Recent reports have also shown that mutations in p53 not only predispose one to cancer, but that the efficacy of chemotherapy is dependent on the presence of functional p53.

Studies of the role of p53 as a tumor suppressor have been complicated because of the fact that p53 has two seemingly opposite functions. First, in response to DNA damaging events, it has the ability to inhibit cell cycle progression at the G1IS border. This has been shown to be accomplished by the ability of p53 to transcriptionally activate the p21 gene. p21 is a potent inhibitor of G1 cyclin dependent kinases. This role of p53 seems logical in that if a cell has suffered a DNA damaging event, replicating the damaged DNA (which occurs in S-phase) could result in propagation of an altered DNA sequence. Therefore, inducing cell cycle arrest before S-phase entry is important to maintain genomic fidelity. In its role as a transcription factor, p53 can also induce genes that are involved in nucleotide excision repair, which are required to repair the DNA damage. In contrast to this protective function of p53, a second function of p53 is to induce apoptosis following DNA damage in certain cell types. This seemingly contradictory function of p53 has been suggested to be important in cases where the incurred DNA damage is beyond repair and, thus, rather than propagate a cell that has undergone mutagenesis p53 by transcriptionally activating genes such as Box (positive regulator of apoptosis) and death receptors such as killer and Fas, it activates a cell suicide program.

Since p53 negatively regulates cell growth by promoting G1 arrest and by inducing apoptosis, it is thought that there is a mechanism in place that suppresses p53 function in dividing tissues. This is accomplished by the proteosame dependent mechanism that ensures that, in actively dividing cells, the level of p53 protein is very low. This proteosome dependent degradation of proteins plays an important role in regulating the proteins involved in activities such as cell cycle progression, cell differentiation, the stress response and apoptosis. Degradation of p53 first requires post-translational ubiquitination of p53 in a series of reactions. MDMZ (mouse double minute 2), a p53 binding protein that has been shown to have ubiquitin ligase activity, plays a major role in the degradation of p53. MDM2 binds to the amino-terminus of p53, which leads to ubiquitination of p53. The C-terminus of p53 has been proposed to be required for proteosome-mediated degradation.

In order for p53 to transactivate target genes in response to a DNA damaging event, its degradation needs to be inhibited so that levels of the protein accumulate within the nucleus and, following activation of DNA binding activity through additional post-translational modification (e.g. phosphorylation), p53 is able to transcriptionally turn on specific genes. In addition, the inhibitory activity of MDM2 must be overcome. This has been proposed to happen by modification of p53 as well as MDM2 such that they fail to interact, thus, preventing p53 ubiquitination and degradation.

Mutations in the tumor suppressor gene p53 are common in human cancer, accounting for 60% of all cancers. Mutations in p53 are quite different from those in most tumor suppressors. The tumor suppressor genes Rb (retinoblastoma) and APC (adenomato-us polyposis coli) are commonly inactivated by nonsense mutations that cause truncation (and, therefore, loss of function) or instability of the protein. But, in p53, more than 90% of the mutations are missense mutations that change the identity of a particular amino acid. Changing the amino acid sequence results in a change in the conformation such that the ubiquitination machinery fails to recognize the protein and thus mutant p53 does not get rapidly degraded, but accumulates. In general, genotoxic stress to cells results in a transient increase in p53 levels, while long term stabilization and accumulation of p53 often results from mutation of the p53 coding sequence.

Sun-exposed skin is heir to three cancer, melanoma, basal cell carcinoma (BCC) and squamous cell carcinoma (SCC). Melanomas, the most deadly, arise in young adults. They begin as a radial proliferation of normally non-proliferating melanocytes. Vertical spread of the lesion can lead to metastasis. SCC and BCC are tumors of keratinocytes, which are cells that routinely proliferate since they are shed upon differentiation into mature keratinocytes. These tumors often appear in the elderly (70 yrs and older) and appear in a background of sun-damaged skin characterized by a loss in elasticity and disordered keratinocytes. Continued sun exposure leads to actinic keratosis which appears as keratinized reddish patches and contain aberrantly differentiating and proliferating cells. These precancers often regress, but one in a thousand progress to SCC. BCC, on the other hand, develops without obvious precursors seemingly from keratinoytes in hair follicles.

Over 90% of the SCCs in patients in the United States have a mutation somewhere in the p53 gene. Many of the same colons are also mutated in internal cancers such as colon or bladder cancer. p53 mutations are also found in skin tumors of experimental mice and p53 knock-out mice are also more prone to develop UV-induced skin cancer. These results demonstrate the importance of p53 mutations in developing skin cancer and the role of p53 as a tumor suppressor gene in the skin. This and the ability to readily (with little invasiveness) analyze epidermal tissue at the genetic, protein and biochemical level led to using skin cancer as a model to study the role of p53 in the biology of cancer using non-invasive imaging of p53.

Because most p53 mutations result in overly-stable p53 protein, staining the epidermis with antibody to normal p53 reveals the presence of cells having mutant p53. These mutant cells typically form a cluster of cells ranging from b0-3000 cells. These clones are obviously more frequent on UV-exposed skin. The clonal arrangement of the p53 mutated cells strongly suggests that they, upon mutation of p53 cells, continue to proliferate and that counting the bomber of clones per cm2 can reveal the approximate mutation frequency. Most clones regress while in some rare instance a clone may suffer a mutagenic hit in an additional gene, which could lead to cancer.

### SUMMARY

In one general aspect, methods of the invention include a process for in vivo monitoring of levels of a chimeric polypeptide. This in vivo monitoring can include the steps of providing a cell, a tissue, an organ or a whole body having a chimeric nucleic acid or a chimeric polypeptide, wherein the chimeric nucleic acid encodes the chimeric polypeptide. The chimeric polypeptide can have a first domain comprising a bioluminescent or a chemiluminescent polypeptide and a second domain comprising a polypeptide of interest. The process further includes expressing the chimeric polypeptide in the cell, tissue, organ or whole body or contacting the cell, tissue, organ or whole body with the chimeric polypeptide and imaging the cell, tissue, organ or whole body to monitor the level of the chimeric polypeptide of interest in the cell, tissue, organ or whole body. In certain embodiments, the image is generated by computer assisted tomography (CAT), magnetic resonance spectroscopy (MRS), magnetic resonance imaging (MRI), positron emission tomography (PET), single-photon emission computed tomography (SPECT), bioluminescence imaging (BLI), or equivalents.

In certain embodiments, the methods of the invention can be used to identify a putative modulator of the amount of the polypeptide in a cell, tissue, organ or whole body by administering a test compound or a test event to the cell, tissue, organ or whole body before, during and/or after expressing the bioluminescent or chemiluminescent chimeric polypeptide and monitoring a change in the level of the polypeptide of interest over at least two time points to -measure a change in the amount of the chimeric polypeptide in the cell, tissue, organ or whole body, thereby identifying the test compound or test event as a modulator of chimeric polypeptide levels, i. e., the polypeptide of interest.

In other aspects, methods of the invention include a process for the in vivo identification of a DNA damaging stimulus or a DNA damaging compound, for the in vivo screening and identification of a test compound or a test stimulus as a putative carcinogen or cell growth modulator, for the in vivo monitoring of the ubiquitin-proteosome pathway, for the in vivo screening and identification of a test compound or a test stimulus as a putative modulator of the ubiquitin-proteasome pathway, for the in vivo screening and identification of a test compound or a test stimulus for modulating ubiquitinase. The methods include providing a cell, a tissue, an organ or a whole body having a chimeric nucleic acid or a chimeric polypeptide, wherein the chimeric nucleic acid encodes the chimeric polypeptide. The chimeric polypeptide of the method has a first domain that is a bioluminescent or a chemiluminescent polypeptide. Exemplary bioluminescent or chemiluminescent compounds include, but are not limited to, a luciferase, an aequorin, an obelin, a mnemiopsin or a berovin. The second domain of the chimeric polypeptide can be a polypeptide that is upregulated or downregulated in response to DNA damage or in response to cell growth, or whose cellular level is regulated by ubiquitination, or is capable of being ubiquitinated.

The test compound or stimulus, which may include a DNA damaging compound or stimulus, can be administered before, during, and/or after expression of the chimeric polypeptide. The methods can further include imaging the cell, tissue, organ or whole body to monitor the level of the polypeptide in the cell, tissue, organ or whole body and noting the change in the level of the polypeptide in response to administration of the compound or stimulus. The image can be generated by computer assisted tomography (CAT), magnetic resonance spectroscopy (MRS), magnetic resonance imaging (MRI), positron emission tomography (PET), single-photon emission computed tomography (SPECT), bioluminescence imaging (BLI), or equivalents.

In certain embodiments, the polypeptides of interest are ones that are capable of being ubiquitinated. The ubiquitinated polypeptide of interest can be degraded by the ubiquitin-proteasome pathway. The polypeptide of interest could potentially be any protein, such as a tumor suppressor protein. Exemplary tumor suppressor proteins can be p53 polypeptide or p73 polypeptide. Examples of a stimulus include, but are not limited to, application of a chemical or a radiation to the cell, tissue, organ, or whole body.

In yet another aspect, the invention includes transgenic, non-human animals having a chimeric nucleic acid, wherein the chimeric nucleic acid comprises an open reading frame operably linked to a promoter, wherein the open reading frame encodes a chimeric polypeptide with a first domain comprising a fluorescent, a bioluminescent or a chemiluminescent polypeptide and a second domain that is upregulated or downregulated in response to DNA damage or in response to cell growth, or whose cellular level is regulated by ubiquitination, or is capable of being ubiquitinated.

In some embodiments, the polypeptide in the transgenic, non-human animal is capable of being ubiquitinated is p53. In other embodiments, the gene encoding an endogenous p53 of the transgenic, non-human animal has been disabled and the animal is incapable of expressing endogenous p53. The transgenic animal may be a mouse.

The present invention further provides a method for in vivo monitoring of levels of a chimeric polypeptide comprising providing a cell, a tissue, an organ or a whole body comprising a chimeric nucleic acid or a chimeric polypeptide, wherein the chimeric nucleic acid encodes the chimeric polypeptide and the chimeric polypeptide comprises a first domain comprising a bioluminescent or a chemiluminescent polypeptide, and a second domain comprising a polypeptide of interest; expressing the chimeric polypeptide in the cell, tissue, organ or whole body or contacting the cell, tissue, organ or whole body with the chimeric polypeptide; and imaging the cell, tissue, organ or whole body to monitor the level of the chimeric polypeptide of interest in the cell, tissue, organ or whole body, wherein the image is generated by computer assisted tomography (CAT), magnetic resonance spectroscopy (MRS), magnetic resonance imaging (MRI), positron emission tomography (PET), single-photon emission computed tomography (SPECT), bioluminescence imaging (BLS) or equivalents. In further embodiments, the polypeptide of interest is capable of being ubiquitinated. In other embodiments, the ubiquitinated polypeptide is degraded by the ubiquitin-proteasome pathway. In even further embodiments, the polypeptide of interest comprises a tumor suppressor protein. In further embodiments, such a tumor suppressor protein comprises a p53 polypeptide or a p73 polypeptide. In yet other embodiments, such methods may further comprise identifying a putative modulator of the amount of the polypeptide in a cell, tissue, organ or whole body by administering a test compound or a test event to the cell, tissue, organ or whole body before, during and/or after the expressing step and monitoring a change in the level of the polypeptide of interest over at least two time points to measure a change in the amount of the bioluminescent or chemiluminescent chimeric polypeptide in the cell, tissue, organ or whole body, thereby identifying the test compound or test event as a modulator of chimeric polypeptide levels.

The present invention also provides a method for in vivo identification of a DNA damaging stimulus or a DNA damaging compound comprising providing a cell, a tissue, an organ or a whole body comprising a chimeric nucleic acid or a chimeric polypeptide, wherein the chimeric nucleic acid encodes the chimeric polypeptide and the chimeric polypeptide comprises a first domain comprising a bioluminescent or a chemiluminescent polypeptide, and a second domain comprising a polypeptide that is upregulated or downregulated in response to DNA damage; providing a test compound or a test stimulus; expressing the chimeric polypeptide in the cell, tissue, organ or whole body or contacting the cell, tissue, organ or whole body with the chimeric polypeptide; administering the compound or stimulus to the cell, tissue, organ or whole body, wherein the administration can be before, during and/or after the expressing step; and imaging the cell, tissue, organ or whole body to monitor the level of the polypeptide in the cell, tissue, organ or whole body, wherein a change in the level of the polypeptide in response to administration of the compound identifies the test compound as a DNA damaging agent; wherein the image is generated by computer assisted tomography (CAT), magnetic resonance spectroscopy (MRS), magnetic resonance imaging (MRI), positron emission tomography (PET), single-photon emission computed tomography (SPECT), bioluminescence imaging (BLS) or equivalents. In further embodiments, the polypeptide upregulated or downregulated in response to DNA damage comprises a tumor suppressor protein. In even further embodiments, such a tumor suppressor protein comprises a p53 polypeptide or a p73 polypeptide. In yet further embodiments, the stimulus comprises administration of a chemical or a radiation to the cell, tissue, organ or whole body.

In further embodiments, the present invention provides a method for in vivo monitoring ubiquitin-proteasome pathway activity, comprising providing a cell, a tissue, an organ or a whole body comprising a chimeric nucleic acid or a chimeric polypeptide, wherein the chimeric nucleic acid encodes the chimeric polypeptide and the chimeric polypeptide comprises a first domain comprising a bioluminescent or a chemiluminescent polypeptide, and a second domain comprising a polypeptide whose cellular levels are regulated by ubiquitination; expressing the chimeric polypeptide in the cell, tissue, organ or whole body or contacting the cell, tissue, organ or whole body with the chimeric polypeptide; and imaging the cell, tissue, organ or whole body to monitor the level of the polypeptide in the cell, tissue, organ or whole body, wherein a change in the level of the polypeptide is an indication of ubiquitin-proteasome pathway activity, wherein the image is generated by computer assisted tomography (CAT), magnetic resonance spectroscopy (MRS), magnetic resonance imaging (MRI), positron emission tomography (PET), single-photon emission computed tomography (SPECT), bioluminescence imaging (BLI), or equivalents.

In other embodiments, the present invention provides a method for in vivo screening and identifying a test compound or a test stimulus as a putative carcinogen or cell growth modulator. In such embodiments, the method comprises providing providing a cell, a tissue, an organ or a whole body comprising a chimeric nucleic acid or a chimeric polypeptide, wherein the chimeric nucleic acid encodes the chimeric polypeptide and the chimeric polypeptide comprises a first domain comprising a bioluminescent or a chemiluminescent polypeptide, and a second domain comprising a polypeptide that is upregulated or downregulated in response to cell growth; providing a test compound or a test stimulus; expressing the chimeric polypeptide in the cell, tissue, organ or whole body or contacting the cell, tissue, organ or whole body with the chimeric polypeptide; administering the compound or stimulus to the cell, tissue, organ or whole body, wherein the administration can be before, during and/or after the expressing step; and imaging the cell, tissue, organ or whole body to monitor the level of the polypeptide in the cell, tissue, organ or whole body, wherein a change in the level of the polypeptide in response to administration of the compound identifies the test compound or the test stimulus as a putative carcinogen or a modulator of cell growth, wherein the image is generated by computer assisted tomography (CAT), magnetic resonance spectroscopy (MRS), magnetic resonance imaging (MRI), positron emission tomography (PET), single-photon emission computed tomography (SPECT), bioluminescence imaging (BLS) or equivalents.

The present invention also provides a method for in vivo screening and identifying a test compound or a test stimulus as a putative modulator of ubiquitin-proteasome pathway activity. In such embodiments, the method comprises providing a cell, a tissue, an organ or a whole body comprising a chimeric nucleic acid or a chimeric polypeptide, wherein the chimeric nucleic acid encodes the chimeric polypeptide and the chimeric polypeptide comprises a fast domain comprising a bioluminescent or a chemiluminescent polypeptide, and a second domain comprising a polypeptide whose cellular levels is regulated by ubiquitination; providing a test compound or a test stimulus; expressing the chimeric polypeptide in the cell, tissue, organ or whole body or contacting the cell, tissue, organ or whole body with the chimeric polypeptide; administering the compound or stimulus to the cell, tissue, organ or whole body, wherein the administration can be before, during and/or after the expressing step; and imaging the cell, tissue, organ or whole body to monitor the level of the polypeptide in the cell, tissue, organ or whole body, wherein a change in the level of the polypeptide in response to administration of the compound identifies the test compound or the test stimulus as a modulator of the ubiquitin-proteasome pathway, wherein the image is generated by computer assisted tomography (CAT), magnetic resonance spectroscopy (MRS), magnetic resonance imaging (MRI), positron emission tomography (PET), single-photon emission computed tomography (SPELT), bioluminescence imaging (BLI), or equivalents.

The present invention also provides a method for in vivo screening and identifying a test compound for modulating ubiquitinase. In such embodiments, the method comprises providing a cell, a tissue, an organ or a whole body comprising a chimeric nucleic acid or a chimeric polypeptide, wherein the chimeric nucleic acid encodes the chimeric polypeptide and the chimeric polypeptide comprises a first domain comprising a bioluminescent or a chemiluminescent polypeptide, and a second domain comprising a polypeptide capable of being ubiquitinated; providing a test compound or a test stimulus; expressing the chimeric polypeptide in the cell, tissue, organ or whole body or contacting the cell, tissue, organ or whole body with the chimeric polypeptide; administering the compound or stimulus to the cell, tissue, organ or whole body, wherein the administration can be before, during and/or after step (c); and imaging the cell, tissue, organ or whole body to monitor the level of the polypeptide in the cell, tissue, organ or whole body, wherein a change in the level of the polypeptide in response to administration of the compound or stimulus identifies the test compound or stimulus as a modulator of ubiquitinase, wherein the image is generated bY computer assisted tomography (CAT), magnetic resonance spectroscopy (MRS), magnetic resonance imaging (MRI), positron emission tomography (PET), single-photon emission computed tomography (SPECT), bioluminescence imaging (BLS) or equivalents.

In further embodiments of the present invention, bioluminescent or chemiluminescent compounds may comprise a luciferase, an aequorin, an obelin, a mnemiopsin or a berovin.

The present invention further provides a transgenic, non-human animal comprising a chimeric nucleic acid, wherein the chimeric nucleic acid comprises an open reading frame operably linked to a promoter, wherein the open reading frame encodes a chimeric polypeptide comprising a first domain comprising a fluorescent, a bioluminescent or a chemiluminescent polypeptide and a second domain comprising a polypeptide capable of being ubiquitinated. In further embodiments, the polypeptide is capable of being ubiquitinated is p53. In even further embodiments, a gene encoding an endogenous p53 in the animal is disabled and the animal is incapable of expressing endogenous p53. In other embodiments, the transgenic, non-human animal is a mouse.

The details of one or more embodiments of the invention are set forth in the accompanying drawings and the description below. Other features, objects, and advantages of the invention is apparent from the description, drawings, and the claims.

### DESCRIPTION OF THE DRAWINGS

FIG. 1A is a schematic diagram of one embodiment of the invention.

FIG. 1B depicts a western blot of p53 accumulation in a sample in response to irradiation as opposed to no accumulation when the sample was not irradiated.

FIG. 2 depicts the results of a western blot of exemplary constructs of the invention in response to DNA damage.

FIG. 3 depicts an example of time dependent accumulation of bioluminescence activity in response to DNA damage.

FIGS. 4A-D depict an example of intracranial glioma growth with MRI imaging while FIGS. 4E-4H depict the growth with BLI imaging.

FIG. 41 describes the color map for the luminescent signal.

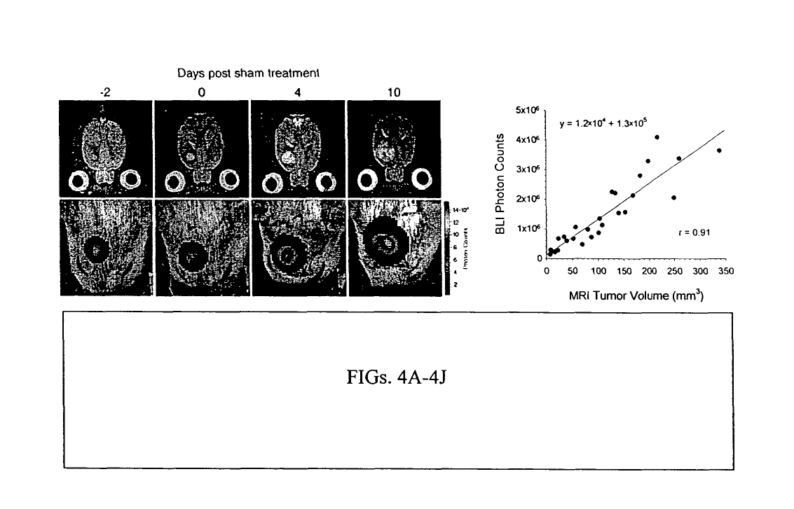

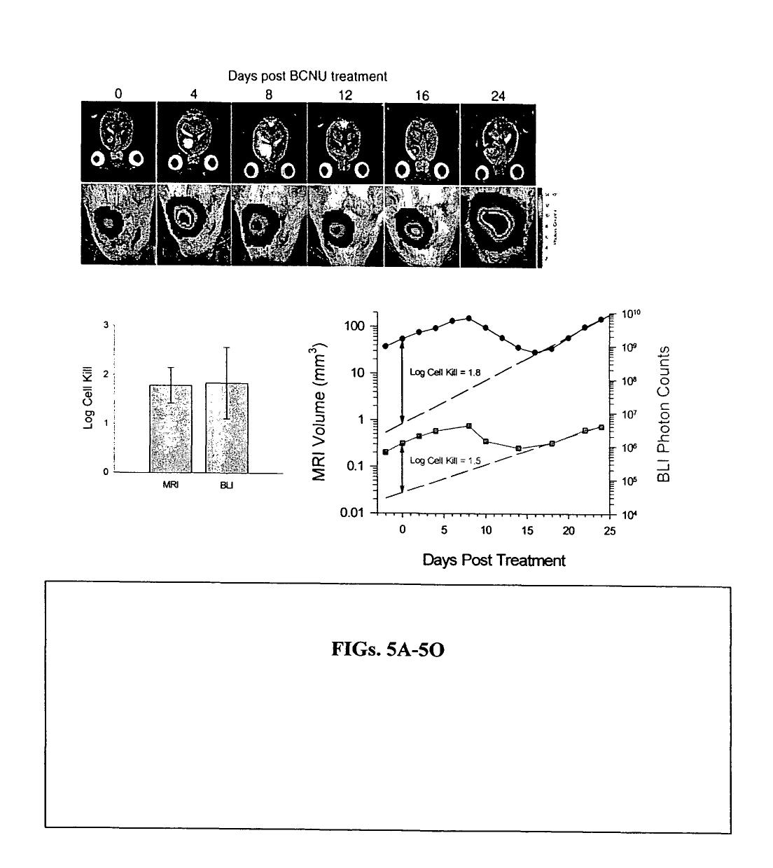

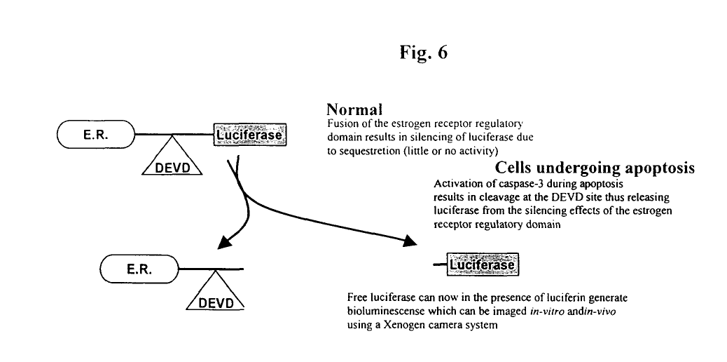

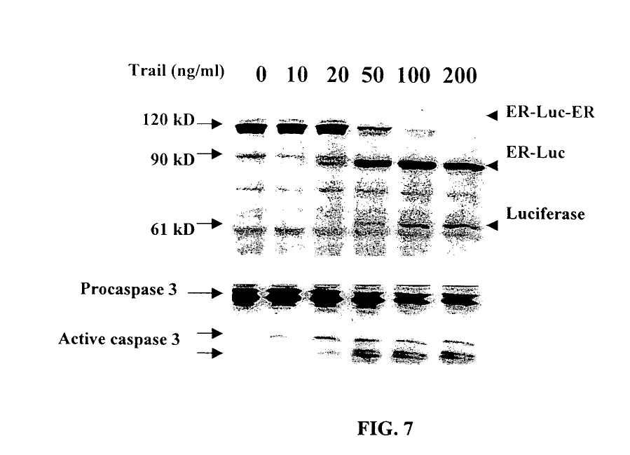

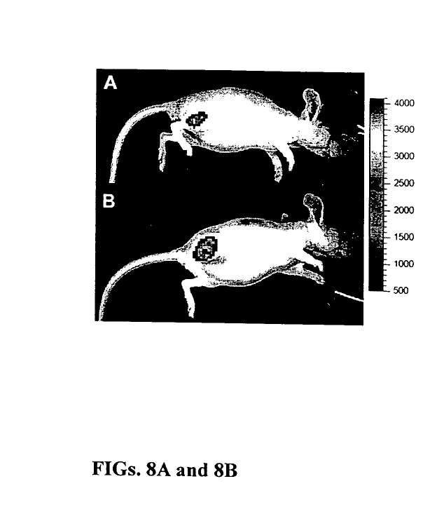

FIG. 4J depicts the correlation of tumor volume with in vivo photon emission

FIGS. 5A-5F depict an example of tumor response to BCNU chemotherapy with MRI imaging while FIGS. 5G-5L depict the response with BLI imaging.

FIG. 5M describes the color map for the photon count.

FIG 5N depicts a bar graph comparing log cell kill values determined from MRI and BLI measurements.

FIG. 5O depicts a quantitative analysis of the tumor progress and response to BCNU chemotherapy.

FIG. 6 is a schematic diagram of a portion of a “knock-in” vector of the present invention.

FIG. 7 depicts a western blot using a luciferase specific antibody showing induction of apoptosis.

FIGS. 8A and 8B depict non-invasive imaging of apoptosis in nude mice.

Like reference symbols in the various drawings indicate like elements.

### DEFINITIONS

Unless defined otherwise, all technical and scientific terms used herein have the meaning commonly understood by a person skilled in the art to which this invention belongs. To facilitate an understanding of the present invention, a number of terms and phrases are defined below:

Ubiquitin functions as a covalent modifier of proteins through the formation of isopeptide bonds between the C-terminal carboxyl group of ubiquitin and the side-chain amino group of a lysine residue of a target protein to form a branched polypeptide. The target protein can be another ubiquitin. The term “palypeptides capable of being ubiquitinated,” as used herein, refers to any protein that can be ubiquitinated, i.e., attachment of ubiquitin to the protein.

Tumor suppressor genes include, but are not limited to, APC, BRCA1, BRCA2, CDH1, CDKN1C, CDKN2A, CYLD, EP300, EXT1, EXT2, MADH4, MAP2K4, MEN1, MLH1, MSH2, NF1, NF2, p53, PRKAR1A, PTCH, PTEN, RB1, SDHD, SMARCB1, STK11, TSC1, TSC2, VHL, and WT1.

Ubiquitinases are proteases that recognize a ubiquitinated protein and can cause its degradation.

The term “DNA damaging compounds” refers to compounds capable of modulating the accumulation of the construct, including proteins and chemicals.

The term “DNA damaging stimuli” refers to those stimuli capable of modulating the accumulation of the construct, including irradiation.

The term “modulation,” with respect to the accumulation of a construct of the invention, includes a 10, 20, 30, 40, 50 fold or more accumulation of the construct upon a DNA damaging event, e.g., contact, directly or indirectly, with a DNA damaging compound and/or DNA damaging stimulus.

As used herein, the term “bioluminescence imaging” or “BLI” includes all bioluminescence, fluorescence or chemiluminescence or other photon detection systems and devices capable of detecting bioluminescence, fluorescence or chemiluminescence or other photon detection systems. Since light can be transmitted through mammalian tissues at a low level, bioluminescent and fluorescent proteins can be detected externally using sensitive photon detection systems; see, e.g., Contag (2000) Neoplasia 2:41-52; Zhang (1994) Clin. Exp. Metastasis 12: 87-92. The methods of the invention can be practiced using any such photon detection device, or variation or equivalent thereof, or in conjunction with any known photon detection methodology, including visual imaging. An exemplary photodetector device is an intensified charge-coupled device (ICCD) camera coupled to an image processor. See, e.g., U.S. Pat. No. 5,650,135. Photon detection devices are manufactured by, e.g., Xenogen (Alameda, Calif.) (the Xenogen IVISTM imaging system); or, Hamamatsu Corp., Bridgewater, N.J.

As used herein, “chimeric” nucleic acid or polypeptide includes any nucleotide or polypeptide sequence having a region not normally found in nature. A chimeric nucleic acid or polypeptide may also have a region of nucleotides or amino acids in locations not normally found in the wildtype sequence.

As used herein, a “computer assisted tomography (CAT)” or a “computerized axial tomography (CAT)” incorporates all computer-assisted tomography imaging systems or equivalents and devices capable of computer assisted tomography imaging. The methods of the invention can be practiced using any such device, or variation of a CAT device or equivalent, or in conjunction with any known CAT methodology. See, e.g., U.S. Pat. Nos. 6,151,377; 5,946,371; 5,446,799; 5,406,479; 5,208,581; 5,109,397. Animal imaging modalities are also included, such as MicroCATTM (ImTek, Inc., Knoxville, Tenn.).

As used herein, “positron emission tomography imaging (PET)” incorporates all positron emission tomography imaging systems or equivalents and all devices capable of positron emission tomography imaging. The methods of the invention can be practiced using any such device, or variation of a PET device or equivalent, or in conjunction with any known PET methodology. See, e.g., U.S. Pat. Nos. 4,151,377; 6,072,177; 5,900,636; 5,608,221; 5,532,489; 5,272,343; 5,103,098. Animal imaging modalities are included, e.g. micro-PETS (Corcorde Microsystems, Inc.).

As used herein, “single-photon emission computed tomography (SPELT) device” incorporates all single-photon emission computed tomography imaging systems or equivalents and all devices capable of single-photon emission computed tomography imaging. The methods of the invention can be practiced using any such device, or variation of a SPELT device or equivalent, or in conjunction with any known SPELT methodology. See, e.g., U.S. Pat. Nos. 6,115,446; 6,072,177; 5,608,221; 5,600,145; 5,210,421; 5,103,098. Animal imaging modalities are also included, such as micro-SPECTs.

As used herein, “magnetic resonance imaging (MRI) device” incorporates all magnetic resonance imaging systems or equivalents and all devices capable of magnetic resonance imaging. The methods of the invention can be practiced using any such device, or variation of an NiRI device or equivalent, or in conjunction with any known MRI methodology. In magnetic resonance methods and apparatus a static magnetic field is applied to a tissue or a body under investigation in order to define an equilibrium axis of magnetic alignment in a region of interest. A radio frequency field is then applied to that region in a direction orthogonal to the static magnetic field direction in order to excite magnetic resonance in the region. The resulting radio frequency signals are detected and processed. The exciting radio frequency field is applied. The resulting signals are detected by radio-frequency coils placed adjacent the tissue or area of the body of interest. See, e.g., U.S. Pat. Nos. 6,151,377; 6,144,202; 6,128,522; 6,127,825; 6,121,775; 6,119,032; 6,115,446; 6,111,410; 602,891; 5,555,251; 5,455,512; 5,450,010; 5,3?8,987; 5,214,382; 5,031,624; 5,207,222; 4,985,678; 4,906,931; 4,558,279. NIItI and supporting devices are manufactured by, e.g., Broker Medical GMBH; Caprius; Esaote Biomedica (Indianapolis, Ilk; Fonar; GE Medical Systems (GEMS); Hitachi Medical Systems America; Intermagnetics General Corporation; Lunar Corp.; MagneVu; Marconi Medicals; Philips Medical Systems; Shimadzu; Siemens; Toshiba America Medical Systems; including imaging systems, by, e.g., Silicon Graphics. Animal imaging modalities are also included, such as micro-MRIs.

As used herein, the terms “computer” and “processor” are used in their broadest general contexts and incorporate all such devices. The methods of the invention can be practiced using any computer I processor and in conjunction with any known software or methodology. For example, a computer/processor can be a conventional general-purpose digital computer, e.g., a personal “workstation” computer, including conventional elements such as microprocessor and data transfer bus. The computer ! processor can further include any form of memory elements, such as dynamic random access memory, flash memory or the like, or mass storage such as magnetic disc optional storage.

As used herein, “bioluminescents” and “chemiluminescents” are polypeptides that can be imaged using techniques known in the art. Polypeptides include all known polypeptides known to be bioluminescent or chemiluminescent, or, acting as enzymes on a specific substrate (reagent), can generate (by their enzymatic action) a bioluminescent or chemiluminescent molecule. They include, e.g., isolated and recombinant luciferases, aequorin, obelin, mnemiopsin, berovin and variations thereof and combinations thereof, as discussed in detail, below. In some aspects, the bioluminescent or chemiluminescent polypeptides are enzymes that act on a substrate that reacts with the reagent in situ to generate a molecule that can be imaged. The substrate can be administered before, at the same time (e.g., in the same formulation), or after administration of the chimeric polypeptidelconstruct (including the enzyme). In other aspects, the bioluminescent or chemiluminescent polypeptides are proteins that can be activated by a stimulating event, such as EM radiation, acoustic energy, or temperature, and imaged. In still other aspects, the bioluminescent or chemiluminescent polypeptides are proteins that can be detected/imaged in its environment without the need for exogenous substrate (reagent) or a stimulating event.

The term “pharmaceutical composition” refers to a composition suitable for pharmaceutical use in a subject (including human or veterinary). The pharmaceutical compositions of this invention are formulations that comprise a pharmacologically effective amount of a composition comprising, e.g., a chimeric composition, a chimeric/recombinant polypeptide, a nucleic acid encoding a chimeric polypeptide of the invention, a vector comprising a nucleic acid of the invention, or a cell of the invention, and a pharmaceutically acceptable carrier. The pharmaceutical formulation of the invention can further comprise a substrate for the bioluminescent or chemiluminescent polypeptide. For example, the chemiluminescent polypeptide can be luciferase and the reagent luciferin. Alternatively, the substrate reagent can be co-administered or administered before or after the chimeric polypeptide formulation.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (also “recombinant protein”) encoded by a recombinant polynucleotide.

The term “nucleic acid” or “nucleic acid sequence” refers to a deoxyribonucleotide or ribonucleotide oligonucleotide, including single- or double-stranded, or coding or non-coding (e.g., “antisense”) forms. The term encompasses nucleic acids, i.e., oligonucleotides, containing known analogues of natural nucleotides. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156.

The term “expression cassette” refers to any recombinant expression system for the purpose of expressing a nucleic acid sequence of the invention in vitro or in vivo, constitutively or inducibly, in any cell, including, in addition to mammalian cells, insect cells, plant cells, prokaryotic, yeast, fungal or mammalian cells. The term includes linear or circular expression systems. The term includes all vectors. The cassettes can remain episomal or integrate into the host cell genome. The expression cassettes can have the ability to self-replicate or not, i.e., drive only transient expression in a cell. The term includes recombinant expression cassettes that contain only the minimum elements needed for transcription of the recombinant nucleic acid.

As used herein the terms “polypeptide,” “protein,” and “peptide” are used interchangeably and include compositions of the invention that also include “analogs,” or “conservative variants” and “mimetics” (e.g., “peptidomimetics”) with structures and activity that substantially correspond to the polypeptides of the invention, including the chimeric polypeptide comprising a bioluminescent or chemiluminescent polypeptide, or a heterologous kinase, and a silencing moiety, and an endogenous protease cleavage motif positioned between the first and second domains. Thus, the terms “conservative variant” or “analog” or “mimetic” also refer to a polypeptide or peptide which has a modified amino acid sequence, such that the changes) do not substantially alter the polypeptide's (the conservative variant's) structure and/or activity (e.g., binding specificity), as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity. Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gln; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that axe conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.”

The terms “mimetic” and “peptidomimetic” refer to a synthetic chemical compound that has substantially the same structural and/or functional characteristics of the polypeptides of the invention (e.g., ability to be specifically recognized and cleaved by enzymes, including proteases). The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetics' structure and/or activity. As with polypeptides of the invention which are conservative variants, routine experimentation will determine whether a mimetic is within the scope of the invention, i.e., that its structure and/or function is not substantially altered. Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclo-hexylcaxbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH2— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357,“Peptide Backbone Modifications,” Marcell Dekker, N.Y.). A polypeptide can also be characterized as a mimetic by containing all or some non-natural residues in place of naturally occurring amino acid residues; non-natural residues are well described in the scientific and patent literature.

### DETAILED DESCRIPTION

The present invention takes advantage of the fact that, in normal cells, ubiquitinated proteins are continually being expressed and, yet, rapidly degraded. Their short half-life is due to ubiquitination and transport to proteosomes for degradation. However, in cases where DNA damage has occurred, degradation of the ubiquitinated protein is prevented, resulting in accumulation of the protein.

For example, in the case of ubiquitinated protein p53, DNA damage can result in the stabilization (accumulation) and activation of p53. Upon accumulation and activation, p53 initiates a cell program to stop dividing (through transcriptional activation of p21, an inhibitor of kinases required for cell cycling). This cell cycle arrest not only prevents the replication of damaged DNA, but also provides an opportunity for the cell to repair the damaged DNA.

In the event that a cell has suffered DNA damage that cannot be repaired, p53 initiates the apoptotic program, thereby, killing the cell that may carry genetic mutations that are not repairable. This function of p53 is crucial for maintaining genetic stability and fidelity and, hence, the title often ascribed to p53 is “guardian of the genome.” Due to the obvious importance of p53 induction in sensing carcinogenic events, the ability to non-invasively image p53 accumulation will provide a powerful tool in evaluating carcinogenic potential within the environment, food, drugs, or other compounds. In addition, it would provide a useful model for evaluating chemopreventative agents.

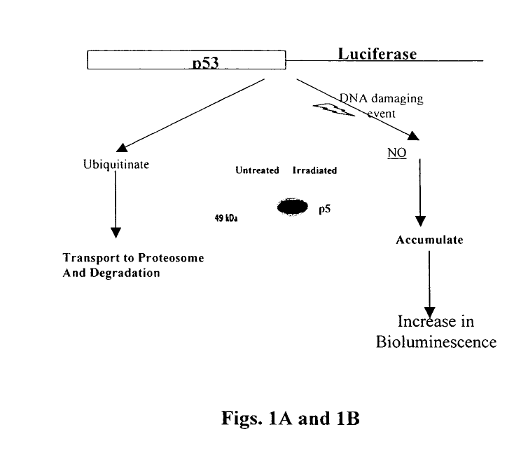

The non-invasive imaging strategy utilizes a physical linkage, such as by gene fusion, of the ubiquitinated protein to a reporter molecule resulting in a chimeric construct. Under normal conditions, the chimeric construct is degraded by the ubiquitin-proteosome pathway. However, in case of DNA damage, the fusion protein will not be degraded, but will instead accumulate, thus, enabling imaging of the construct for monitoring levels of the reporter. For example, a construct whereby p53 is fused to luciferase can be used to practice the method of the invention, see FIG. 1A. Under normal conditions, the p53-Luc construct is degraded by the ubiquitin-proteosome pathway. However, upon DNA damage, the p-53-Luc construct will not become degraded, and will, instead, accumulate, for example about 10 to 50 fold or more, thus, enabling imaging of the p53 activation by monitoring the level of luciferase activity using bioluminesensce imaging. FIG. 1B depicts a western blot showing p53 accumulation in response to ionizing radiation and no accumulation when not subjected to ionizing radiation, i.e., a DNA damaging event.

Accordingly, the invention provides chimeric polypeptides (also known as constructs), nucleic acids encoding the chimeric polypeptides and methods for using them to non-invasively image polypeptides capable of being ubiquitinated in vivo. Polypeptides capable of being ubiquitinated include tumor suppressor proteins. The non-invasive imaging can be performed on cells, tissues, organs and whole bodies.

Because many tumor suppressor genes are specifically associated with certain normal and abnormal conditions and diseases, such as cell death (e.g., apoptosis), cancer, infections and other conditions, in vivo imaging of tumor suppressor gene (e.g., p53) inactivation is useful for identifying, targeting, diagnosing, and the like. The imaging can be by computer assisted tomography (CAT), magnetic resonance spectroscopy (MRS), magnetic resonance imaging (MRI), positron emission tomography (PET), single-photon emission computed tomography (SPECT), or bioluminescence imaging (BLI).

Accordingly, it is desirable to develop a strategy that enables non-invasive imaging of p53 activation in response to genotoxic stress (carcinogenesis) as well as detect for the presence of mutant p53. One strategy is to use constructs, such as a p53-Luciferase fusion as a reporter for p53 levels, and conduct tests using the constructs with tissue culture cells. The optimal construct will then be tested in-vivo using a tumor xenograft model. A genetically engineered mouse can also be generated (knock-in), wherein the genomic p53 locus has been altered to express the desired construct, e.g., p53-Luciferase fusion protein.

Bioluminescent or Chemiluminescent Polypeptides

The invention provides a chimeric polypeptide, e.g., a recombinant polypeptide, and a pharmaceutical composition, comprising a bioluminescent or chemiluminescent polypeptide. As defined above, these polypeptides include enzymes that act on a specific reagent to generate a molecule that can be imaged. The reagent can be an exogenously introduced compound (e.g., luciferase reacting with luciferin in situ), or the reagent can be an endogenous compound normally found in the environment where the polypeptide is expressed. Additionally or altematively, the polypeptides can include a portion that can be imaged directly.

In alternative aspects, these polypeptides include, e.g., luciferase, aequorin, halistaurin, phialidin, obelin, mnemiopsin or berovin, or, equivalent photoproteins, and combinations thereof. The compositions and methods of the invention also include recombinant forms of these polypeptides as recombinant chimeric or “fusion” proteins, including chimeric nucleic acids and constructs encoding them. Methods of making recombinant forms of these polypeptides are well known in the art, e.g., luciferase reporter plasmids are described, e.g., by Everett (1999) J. Steroid Biochem. Mol. Biol. 70:197-201. Sala-Newby (1998) Immunology 93:601-609, described the use of a recombinant cytosolic fusion protein of firefly luciferase and aequorin (luciferase-aequorin). The Ca²⁺-activated photoprotein obelin is described by, e.g., Dormer (1978) Biochim. Biophys. Acta 538:87-105; and, recombinant obelin is described by, e.g., Illarionov (2000) Methods Enzymol. 305:223-249. The photoprotein mnemiopsin is described by, e.g., Anctil (1984) Biochem J. 221:269-272. The monomeric Ca²⁺-binding protein aequorin is described by, e.g., Kurose (1989) Proc. Natl. Acad. Sci. USA 86:80-84; Shimomura (1995) Biochem. Biophys. Res. Common. 211:359-363. The aequorin-type photoproteins halistaurin and phialidin are described by, e.g., Shimomura (1985) Biochem J. 228:745-749. Ward (1975) Proc. Natl. Acad. Sci USA 72:2530-2534, describes the purification of mnemiopsin, aequorin and berovin. The recombinant bioluminescent or chemiluminescent chimeric polypeptides of the invention can be made by any method, see, e.g., U.S. Pat. No. 6,087,476, that describes mfg recombinant, chimeric luminescent proteins. U.S. Pat. Nos. 6,143,50; 6,074,859; 6,074,859, 5,229,285, describe making recombinant luminescent proteins. The bioluminescent or chemiluminescent activity of the chimeric recombinant polypeptides of the invention can be assayed, e.g., using assays described in, e.g., U.S. Pat. Nos. 6,132,983; 6,087,476; 6,060,261; 5,866,348; 5,094,939; 5,744,320. Various photoproteins that can be used in compositions of the invention are described in, e.g., U.S. Pat. Nos. 5,648,218; 5,360,728; 5,098,828.

In Vivo Bioluminescent Imaging

The invention provides compositions and methods to enhance the imaging of cells and tissues by, e.g., bioluminescence imaging (BLI). In vivo Bioluminescent Imaging (BLI) is a relatively new imaging modality; see discussion above and, e.g., Contag (2000) Neoplasia 2:41-52. This modality consists of the detection of a photoprotein (i.e., an optical reporter), such as luciferase from the firefly, using a sensitive photon detection system. The number of photons emitted from cells expressing the photoprotein (e.g., luciferase) can be quantitatively detected and overlayed (projected) onto a visual picture of the animal (including humans). This imaging approach provides a two-dimensional image data set and thus provides some spatial information as to the origin of the signal within the animal. An exciting aspect of BLI is its excellent sensitivity along with its ability to report on “molecular events” using specifically designed luciferase reporter constructs.

Nanoparticles and imaging of Brain Tumors

The invention provides pharmaceutical formulations comprising the chimeric polypeptides of the invention that can further comprise imaging contrast agents (see, e.g., U.S. Pat. No. 4,731,239). The pharmaceutical formulations and/or the contrast agents can be administered by nanoencapsulation, e.g., by hydrogel nanoparticles (and liposomes, which are discussed below). Nanoencapsulation can be used to manipulate the environment surrounding the pharmaceutical formulation and/or the contrast agent. Although the contrast between healthy and abnormal tissues is strong, there exists considerable overlap of magnetic resonance imaging (MRI) T₁ and T₂ signals in all tissues. This physical property of biological tissues renders necessary the use of contrast agents for adequate resolution of many lesions: in particular, the diffuse margins of some lesions. Contrast agents for magnetic resonance imaging typically affect the protons on adjacent water molecules shortening either the T₁ or T₂ signals generated in the magnetic field. The most important factor in enhancement of relaxation is the difference between T₁ and T₂. There must be direct contact between protons and the magnetic parts of the contrast agent in order to shorten the T₁ component significantly. This effect can be clearly observed when gadolinium chelates are encapsulated in liposomes with resulting weakening of the T₁ signal. Weakening of the T₁ signal is thought to be due to the reduced access of water to the cavity of the liposome.

Enhancement of T₂ effects, however, requires clustering of the contrast agent and proximity to each other. This clustering of magnetic contrast agent exerts a greater influence over a much larger localized field. Thus, incorporation into liposomes increases the proximity of T₂ contrast agents and enhances their effectiveness. Incorporation of contrast agents into the body of hydrogel nanoparticles has with it the potential advantages of both immobilizing and clustering the contrast agent and providing a material through which water can freely diffuse.

The pharmaceutical compositions of the invention can further comprise monocrystalline iron oxide nanoparticles (MION), which have been successfully used in a variety of biological and clinical applications. MION has an average diameter of approximately 18 to 24 nm and thus are able to penetrate endothelial fenestrations throughout the body and are cleared through the reticuloendothelial system and are disposed of by hepatic metabolism of iron. MION has excellent contrast characteristics in viva and out-performs the most effective dendrimer-conjugated contrast agents.

Polypeptides and Peptides

The invention provides a chimeric polypeptide comprising a bioluminescent or chemiluminescent domain, and a second domain comprising a polypeptide capable of being ubiquitinated. As noted above, the term polypeptide includes peptides and peptidomimetics, etc. Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art.

Polypeptides and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Bangs, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer). The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman, et al. (Eds) John Wiley & Sons, Inc., N.Y. Polypeptides incorporating naimetics can also be made using solid phase synthetic procedures, as described, e.g., by Di Marchi, et al., U.S. Pat. No. 5,422,426. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234. Modified peptides of the invention can be further produced by chemical modification methods, see, e.g., Belousov (1997) Nucleic Acids Res. 25:3440-3444; Frenkel (1995) Free Radic. Biol. Med. 19:373-380; Blommers (1994) Biochemistry 33:7886-7896.

Peptides and polypeptides of the invention can also be synthesized and expressed as chimeric or “fusion” proteins with one or more additional domains linked thereto for, e.g., to more readily isolate a recombinantly synthesized peptide, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Core, Seattle Wash.). The inclusion of a cleavable linker sequences such as Factor Xa or enterokinase (Invitrogen, San Diego Calif.) between the purification domain and GCA-associated peptide or polypeptide can be useful to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-14). The histidine residues facilitate detection and purification while the enterokinase cleavage site provides a means for purifying the epitope from the remainder of the fusion protein.

Nucleic Acids and Expression vectors

This invention provides nucleic acids encoding the chimeric polypeptides of the invention. As the genes and expression cassettes (e.g., vectors) of the invention can be made and expressed in vitro or in vivo, the invention provides for a variety of means of making and expressing these genes and vectors. One of skill will recognize that desired phenotypes can be obtained by modulating the expression or activity of the genes and nucleic acids (e.g., promoters) within the expression cassettes of the invention. Any of the known methods described for increasing or decreasing expression or activity can be used for this invention. The invention can be practiced in conjunction with any method or protocol known in the art, which are well described in the scientific and patent literature.

The nucleic acid sequences of the invention and other nucleic acids used to practice this invention, whether RNA, eDNA, genomic DNA, expression cassettes, vectors, viruses or hybrids thereof, may be isolated from a variety of sources, genetically engineered, amplified, and/or expressed recombinantly. Any recombinant expression system can be used, including, in addition to bacterial cells, e.g., mammalian, yeast, insect or plant cell expression systems.

Alternatively, these nucleic acids can be synthesized in vitro by well-known chemical synthesis techniques, as described in, e.g., Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques far the manipulation of nucleic acids, such as, e.g., generating mutations in sequences, subcloning, labeling probes, sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Transgenic non-human animals

The invention provides transgenic, non-human animals, e.g., goats, rats and mice, comprising the chimeric nucleic acids of the invention. These animals can be used, e.g., as in vivo models to study apoptosis, or, as models to screen for enzyme activity in vivo. For example, an increase in the activity of an enzyme capable of cleaving the endogenous protease cleavage domain on the in vivo produced chimeric polypeptide can be read by BLI, PET, MRI, etc. Transgenic, non-human animals are excellent models for imaging apoptosis in vivo by determining the activity of apoptosis-associated enzymes. The coding sequences for the chimeric polypeptides can be designed to be constitutive, or, under the control of tissue-specific, developmental-specific or inducible transcriptional regulatory factors.

Transgenic, non-human animals can be designed and generated using any method known in the art; see, e.g., U.S. Pat. Nos. 6,156,952; 6,118,044; 6,111,166; 6,107,541; 5,959,171; 5,922,854; 5,892,070; 5,880,327; 5,891,698; 5,639,940; 5,573,933, describing making and using transgenic mice, rats, rabbits, sheep, pigs and cows. See also, e.g., Pollock (1999) J. Immunol. Methods 231:147-157, describing the production of recombinant proteins in the milk of transgenic dairy animals; Baguisi (1999) Nat. Biotechnol. 17:456-461, demonstrating the production of transgenic goats.

Formulation and Administration Pharmaceuticals

The invention provides pharmaceutical formulations comprising the chimeric molecules of the invention and a pharmaceutically acceptable excipient suitable for administration to image in vivo constructs of the invention, and methods for making and using these compositions. Pharmaceutical compositions comprising enzymes for imaging the chimeric molecules are also contemplated in the present invention. These pharmaceuticals can be administered by any means in any appropriate formulation. Routine means to determine drug regimens and formulations to practice the methods of the invention are well described in the patent and scientific literature. For example, details on techniques for formulation, dosages, administration and the like are described in, e.g., the latest edition of Remington's Pharmaceutical Sciences, Maack Publishing Co, Easton Pa.

The formulations of the invention can include pharmaceutically acceptable carriers that can contain a physiologically acceptable compound that acts, e.g., to stabilize the composition or to increase or decrease the absorption of the pharmaceutical composition. Physiologically acceptable compounds can include, for example, carbohydrates, such as glucose, sucrose, or dextrans, antioxidants, such as ascorbic acid or glutathhione, chelating agents, low molecular weight proteins, compositions that reduce the clearance or hydrolysis of any coadministered agents, or excipients or other stabilizers and/or buffers. Detergents can also used to stabilize the composition or to increase or decrease the absorption of the pharmaceutical composition. Other physiologically acceptable compounds include wetting agents, emulsifying agents, dispersing agents or preservatives that are particularly useful for preventing the growth or action of microorganisms. Various preservatives are well known, e.g., ascorbic acid. One skilled in the art would appreciate that the choice of a pharmaceutically acceptable carrier, including a physiologically acceptable compound defends, e.g., on the route of administration and on the particular physio-chemical characteristics of any co-administered agent.

In one aspect, the composition for administration comprises a chimeric polypeptide of the invention in a pharmaceutically acceptable carrier, e.g., an aqueous carrier. A variety of carriers can be used, e.g., buffered saline and the like. These solutions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and is selected primarily based on fluid volumes, viscosities, body weight and the like in accordance with the particular mode of administration and imaging modality selected.

The pharmaceutical formulations of the invention can be administered in a variety of unit dosage forms; depending upon the particular enzyme-expressing cell or tissue or cancer to, be imaged, the general medical condition of each patient, the method of administration, and the like. Details on dosages are well described in the scientific and patent literature, see, e.g., the latest edition of Remington's Pharmaceutical Sciences. The exact amount and concentration of chimeric polypeptide or pharmaceutical of the invention and the amount of formulation in a given dose, or the “effective dose” can be routinely determined by, e.g., the clinician/technician. The “dosing regimen,” will depend upon a variety of factors, e.g., whether the enzyme expressing cell or tissue or tumor to be image is disseminated or local, the site of application, the general state of the patient's health, age and the like. Using guidelines describing alternative dosaging regimens, e.g., from the use of other imaging contrast agents, the skilled artisan can determine by routine trials optimal effective concentrations of pharmaceutical compositions of the invention. The invention is not limited by any particular dosage range.

The pharmaceutical compositions of the invention (e.g., chimeric polypeptides) can be delivered by any means known in the art systemically (e.g., intravenously), regionally, or locally (e.g., infra- or peri-tumoral or intracystic injection, e.g., to image bladder cancer) by; e.g., intraarterial, intratumoral, intravenous (IV), parenteral, infra-pleural cavity, topical, oral, or local administration, as subcutaneous, infra-tracheal (e.g., by aerosol) or transmucosal (e.g., buccal, bladder, vaginal, uterine, rectal, nasal mucosa), infra-tumoral (e.g., transdermal application or local injection). For example, infra-arterial injections can be used to have a “regional effect,” e.g., to focus on a specific organ (e.g., brain, liver, spleen, lungs), for example, infra-hepatic artery injection or infra-carotid artery injection. If it is desired to deliver the preparation to the brain, it can be injected into a carotid artery or an artery of the carotid system of arteries (e.g., occipital artery, auricular artery, temporal artery, cerebral artery, maxillary artery, etc.).

The pharmaceutical formulations of the invention can be presented in unit-dose or mufti-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Therapeutic compositions can also be administered in a lipid formulation, e.g., complexed with liposomes or in lipid/nucleic acid complexes or encapsulated in liposomes, as in immunoliposomes directed to specific cells. These lipid formulations can be administered topically, systemically, or delivered via aerosol. See, e.g., U.S. Pat. Nos. 6,149,937; 6,146,659; 6,143,716; 6,133,243; 6,110,490; 6,083,530; 6,063,400; 6,013,278; 5,958,378; 5,552,157.

The invention provides kits comprising the compositions, e.g., the pharmaceutical compositions, chimeric polypeptides, nucleic acids, expression cassettes, vectors, and cells of the invention, to image the constructs. The kits also can contain instructional material teaching methodologies, e.g., how and when to administer the compositions, how to apply the compositions and methods of the invention to imaging systems, e.g., computer assisted tomography (CAT), magnetic resonance spectroscopy (MRS), magnetic resonance imaging (MRI), positron emission tomography (PET), single-photon emission computed tomography (SPECT) or bioluminescence imaging (BLI). Kits containing pharmaceutical preparations (e.g., chimeric polypeptides, expression cassettes, vectors, nucleic acids) can include directions as to indications, dosages, routes and methods of administration, and the like.

### EXAMPLES

### Example 1

### Construction of p53-luciferase Fusions with Luciferase at the Amino- or Carboxyl-terminus of p53

To ensure that all p53 functions (ability to induce p21 and cell cycle arrest or apoptosis) and biochemical characteristics (half life, cellular localization) were retained, two fusion molecules, one that has luciferase at the amino- (Luc-p53) and a second that has the luciferase at the carboxy- terminus (p53-Luc), were constructed and analyzed.

The p53-luciferase (p53-Luc) construct was made using the following primers:

(1) 5-prime p53: ggaattc aagctt cactgcc atg gag gag ccg cag gtc (SEQ ID NO:1) (the underlined sequence codes for the first 6 amino acids of p53);

(2) 3-prime p53: ttt ctt tat gtt ttt ggc gtc ttc-gtc tga gtc agg ccc ttc tgt (SEQ ID NO:2) (this is an antisense oligo; the underlined sequence would prime the last 7 aa of p53);

(3) 5-prime luciferase: aca gaa ggg cct gac tca gac-gaa gac gcc aaa aac ata aag aaa (SEQ ID NO:3) (the underlined sequence codes for the first 7 aa of luciferase); and

(4) 3-prime luciferase: gaattc gctagc tta cac ggc gat ctt tcc gcc ctt (SEQ ID NO:4) (this is an antisense oligo; the underlined sequence codes for the last 7 codons of luciferase).

The p53 sequence was amplified by PCR using primers 1 and 2, while luciferase was amplified using primers 3 and 4. The resulting fragments were purified, and 20 ng of each was used in a second PCR reaction using primers 1 and 4. Since the 3-prime sequence of the p53 fragment and the 5-prime sequence of the luciferase have complimentary bases, over 45 bp (see primers 2 and 3) the two PCR fragments “join” to generate a single PCR product, which codes for the fusion protein. This sequence can then be cloned using the EcoRI or HindIII sites at the 5-prime end (see primer 1) or EcoRi and NheI at the 3-prime end (see primer 4).

The primer design and cloning strategy for the Luc-p53 is analogous to the p53-Luc design provided above.

Biochemical Characterization of p53-Luc and Luc-p53

In some embodiments, the fusion proteins are characterized to assess similarity to wild type p53 with regard to their ability to become ubiquitinated, their half life, and reactivity with antibodies specific for wild type p53, but not mutant p53.

Wild type p53 and the two fusions are transfected into HCT11b (p53−/−; obtained from the lab B. Vogelstein, Yale) cells and 48 hr after transfection the cellular p53 are immunoblotted with a p53 specific antibody or a luciferase antibody. These transfections are done with or without co-transfection with the MDM2 expression vector (since it may be limiting in the absence of p53). Ubiquitination is detected as a ladder of bands due to an increase in molecular weight (detailed protocol described in Maki et al., 2000).

As an alternate but more quantitative strategy, immunoprecipitation of p53 using a p53 specific antibody or the luciferase antibody from cells transfected with wild type p53 and the two fusions are performed. These immunoprecipitates are resolved using 8% SDS-PAGE and then blotted onto a membrane. This membrane is then probed with a ubiquitination specific antibody (Sigma). The level of staining is directly proportional to the level of ubiquitination.

The half lives of the wild type molecule compared to the two fusions are determined 48 hr after transfection, the cells are labeled for 15 mins. with ³⁵S-Met and Cys and then the unincorporated label is extensively washed. In addition, excess cold Met and Cys are added to ensure that no additional incorporation of radiolabel occurs in the cells. Extracts from the labeled cells are prepared at various times (15, 30, 45, 60 and 90 mins. after labeling) and used to immunoprecipitate p53. The extract are analyzed by SDS-PAGE and autoradiography. This reveals the approximate half life (when approximately half the counts have disappeared from the p53 or p53-Luc band). Typically, p53 has a half life of approximately 30-40 mins.

In addition to ubiquitination, p53 also undergoes phosphorylation and acetylation post-translationally. As above, the p53 from cells transfected with wild type p53 and the two fusions are immunoprocipitated using a p53 specific, antibody, resolved on a gel and western blotted using an antibody specific for a phospho-serine at residue 15 (NEB) as well as an antibody specific for an acetyl group at Lys 382 (Oncogene).

Immunoprecipitation experiments from radio-labeled cells expressing wild type and the two fusions are also performed. The antibodies are specific for a conformation present only on wild type p53 but not mutant p53, as well as vice-versa. For e.g., the mAb 1620 only recognizes the wild type conformation while mAb 240 only recognizes the mutant conformation.

Functional Characterization of u53 Luc and Luc a53

In some embodiments, the fusion proteins are further tested to confirm whether they have retained the functions of p53 and luciferase—for example, whether, like p53, the fusion proteins have the capacity to transcriptionally activate a p53 responsive reporter construct (MDM2-Luc) or whether the overexpression of the fusion proteins results in cell cycle arrest and/or apoptosis as in the case of wild type p53.

Using a reporter wherein the MDM-2 promoter drives transcription of the Luc gene, the ability of the wild type and fusion proteins to transactivate a p53 responsive promoter is investigated. HCT116 (p53−/−) is co-transfected with a plasmid, wherein the CMV promoter constitutively drives expression of LacZ (as a control for transfection efficiency), as well as the reporter and one of the p53 constructs. 48 hr after transfection, the cells are lysed and the amount of LacZ activity and luciferase activity determined (Promega). This is done with and without a DNA damaging event (irradiation with 5 Gy). Like wild type p53, the fusions also transactivate the MDM-2 promoter and that irradiation enhances the level of transactivation.

Use of p53-Luc or Luc-p53 as Reporters for DNA Damaging Events

The treatment of a stable cell line expressing p53-Luc or Luc-p53 with a DNA damaging agent (i.e. ionizing radiation or chemotherapeutic agents) is investigated to see if it results in stabilization of the fusion proteins (decreased ubiquitination, increased half life and increased steady state levels), as indicated by a corresponding increase in bioluminescence activity. This investigation can be used to determine the dose responsiveness of the accumulation of p53 (by westerns) and luciferase (westerns and bioluminescence).

As previously discussed, stable cell lines using the p53 knock-out line HCT116 p53−/− obtained from the Vogelstein lab (Yale, Conn.) were constructed. Cell lines derived from each of the three constructs (wildtype, p53-Luc and Luc-p53) are treated with various doses of ionizing radiation (0, 2, 4,8 and 10 Gy) and at various times (0,1, 2, 4, 8 and 24 hrs) the cells are analyzed for (a) p53 (or fusion) accumulation by western blot analysis, (b) increase in bioluminescence due to accumulation of the fusion after DNA damage, and (c) functional activation of p53 by doing flow analysis after propidium iodide staining. This last assay indicates whether, in response to DNA damage, a cell cycle arrest (GI or G2) and/or apoptosis is induced.

In-vivo Imaging of p53 Induction in Response to a DNA Damaging Event.

In some embodiments, imaging of p53 induction in stable cell lines expressing p53-Luc or Luc-p53 when grown as xenograft tumors is conducted. Stable cell lines (expressing the above described p53 fusions) are implanted into nude mice as xenografts. Upon establishment of the tumors, the mice are treated with UV irradiation to induce DNA damage and the response to this agent is monitored by bioluminescence imaging and by immunohistochemistry of frozen sections before and after treatment (using a luciferase and/or p53 antibody). A time-course of the induction is determined by immunohistochemistry and bioluminescence imaging, and their correlation is determined.

In other embodiments, studies initiated on tumors of approximately 100 mm³ in volume are conducted. Animals are divided into 7 groups (6 animals/group), consisting of control and UV treated using a UVB lamp at a dose of 2,000 J/m². BLI is conducted at 0, 2, 4, 8, 12, 16, and 24 hours. For each BLI session, a single i.p. injection of a luciferin dose at 150 mg/kg is administered 15 minutes prior to imaging. Data is collected in 1 minute acquisitions and stored for quantitative image analysis. Photon counts are quantitated for each tumor over time and data from each treatment group are summed and the average (+/−SD) values plotted versus time. Statistical analysis between these groups is accomplished at each time point using student t test. In addition, 5 mm slices are cut from snap frozen dorsal skin biopsies and placed on slides. Air dried slides are fixed in acetone for 10 min and then washed in PBS and blocked in 5% goat serum. Using a p53 specific antibody as well as a luciferase specific antibody, cells undergoing p53 activation are identified and quantified by microscopic examination. This enables direct correlation of photon counts to number of p53 positive cells.

Based upon in vitro results, it is contemplated that p53 levels accumulate approximately 4 hours after UV irradiation, after which they decline over time. This is mirrored by photon counts using BLI.

Optimization of the Sensitivity of the Imaging Strategy

Dose response experiments to determine the sensitivity of the reporter system as well as to determine the dose response relationships of bioluminescensce activity and amount of fusion protein by immunohistochemistry to dose of DNA damaging agent is determined. Briefly, xenograft tumors are treated with various doses of UV irradiation and the response of the tumor to this treatment is measured by an increase in bioluminescense activity and by measurement of fusion protein levels after resection of the tumors (imunohistochemistry).

In addition experiments are performed where instead of varying the time, the dose of UV is varied. Mice (6 animals/group 5 doses of irradiation, including a control group at 2 different time points) receive UVB doses of 40, 250, 500, and 2,000 J/m² at an optimal time (found to provide for maximal p53 activation). The animals are imaged using BLI. In addition, 5 mm slices are cut from snap frozen dorsal skin biopsies and placed on slides. Air dried slides are fixed in acetone for 10 min and then washed in PBS and blocked in 5% goat serum. Using a p53 specific antibody as well as a luciferase specific antibody, cells undergoing p53 activation are identified and quantified by microscopic examination. This enables direct correlation of photon counts to number of p53 positive cells.

### Example 2

### Accumulation of p53-Luc Protein in Response to DNA Damage

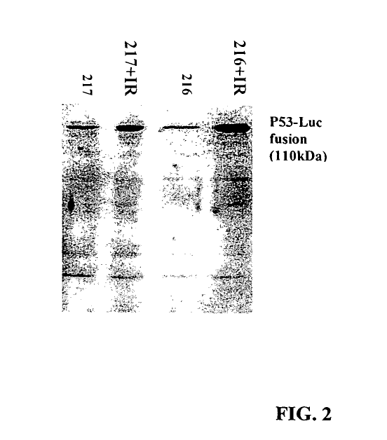

FIG. 2 depicts a western blot showing accumulation of a composition of the invention in response to DNA damage. The exemplary construct is a fused protein wherein the last sense codon of p53 is fused to the first codon of luciferase. The recombinant DNA molecule was stably transfected into MCF-7 cells and the resulting clones were analyzed for expression of the fusion polypeptide (53 kDa of p53 and 60 kDa of luciferase equals 113 kDa) using a luciferase specific antibody. As shown in FIG. 2, while a significant level of the fusion protein was detected in the absence of a DNA damaging event, the two independent stable cell lines (clones 216 and 217) showed reproducible and significant increases in the levels of the p53-Luc fusion protein (217 vs. 217+IR and 216 vs. 216) after irradiation. This indicates that similar to wildtype p53, the p53-Luc fusion is stabilized in response to DNA damaging events.

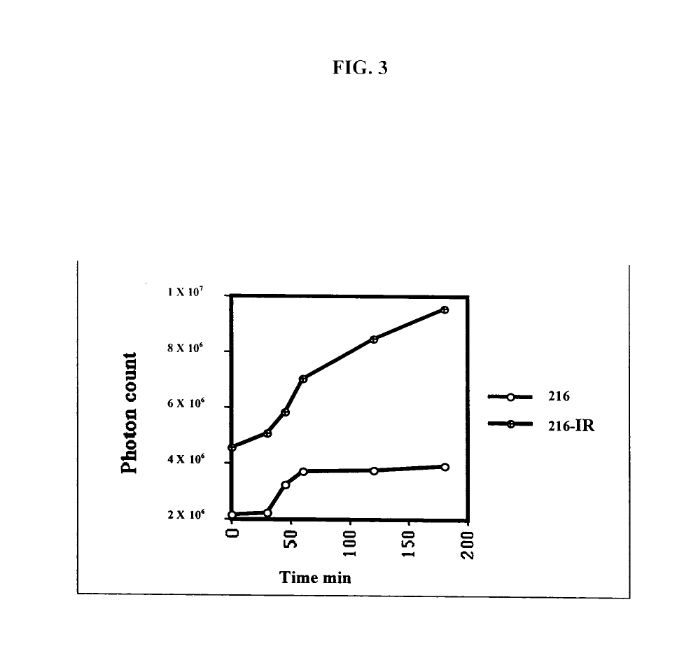

FIG. 3 depicts the time dependent accumulation of bioluminescence activity in response to a DNA damaging event. Clone 216 was imaged at multiple times in the absence (216) and presence (216−IR) of ionizing radiation pre-treatment. As shown in the graph, irradiation results in a steady increase in bioluminescence activity. This data is consistent with the previous figure wherein the p53-Luc protein was shown to accumulate. As seen here, there is an approximately 4-5× increase in bioluminescence in response to irradiation. It should be noted that these experiments were done using a stable cell line wherein the fusion is being expressed constitutively from a robust promoter (adenovirus major late promoter), which is why there is significant amount of protein and bioluminescence in the absence of DNA damage. It is anticipated that when the experiments are carried out in a “knock-in” mouse, the background levels of the fusion are as low as that of p53 (see the western blot depicted in FIG. 1) and, therefore, the signal to noise are much improved.

### Example 3

### Construction of a “Knock-in” Mouse that Expresses a p53-Luc Fusion Instead of p53

A recombinant DNA construct enables the “Knock-in” of the luciferase coding sequence into the p53 gene. Exon 11 of p53 codes for the last 26 amino acids of the p53 gene as well as the stop codon and 3-prime non-coding sequences. The exon 11 sequence is replaced (knock-in) with another sequence that, for example, codes for the last 26 amino acids of p53 followed by the luciferase coding sequence and a stop codon. This replacement results in mice that express the p53-Luc fusion from the authentic p53 promoter and, therefore, the fusion is regulated transcriptionally and post-transcriptionally as if it were wild type p53. Prior to its use in the generation of mutant mice, sequence analysis of all coding exons is performed to ensure that mutations within the p53 sequence are not present (e.g. as a result of PCR).

From ES cell DNA, using long range PCR, a BamHI-PstI fragment which spans intron 6, exon 7, intron 7, exon 8, intron 8, exon 9 and intron 9 is amplified. Subsequently, exons 10 and 11 contained within a PstI-PvuII fragment are amplified. This fragment is used to alter the exon 11 sequence such that it codes for the last 26 amino acids of p53 (no stop codon), as well as the complete Luciferase coding sequence. A third fragment is amplified contained on a PvuII-EcoRI fragment. The three fragments (BamHI-Pstl, Pstl-PvuII, PvuII-EcoRI) are assembled together. Into this composite, a pGK neo cassette is inserted at the PstI site and an EcoRI-BamHI fragment containing the HSV-TK expression cassette is inserted at the 5-prime end. The resulting EcoRI-EcoRI fragment is purified and transfected into ES cells for selection of homologous recombinants.

Generation of “Knock-in” ES Cells and Mice

ES cells cultured in the lab using standard techniques, as set forth in Maniatis or any other laboratory manual, are electrotraporated with the linearized and purified transfer vector after which they are selected for neo-resistance and gancyclovir resistance. The resulting clones are selected and scaled up for further analysis. This analysis includes PCR to quickly screen multiple clones, after which the selected clones are processed for southern blot analysis. The southern blots differentiate clones that have a homologous recombination event leading to a larger exon 11 from clones that are neo-resistant due to nonhomologous events. These activities are performed in collaboration with the transgenic core.

Expression of the fusion polynucleotide in place of the wild type p53 sequence is confirmed by northern blot analysis using a probe specific for the fusion and not the endogenous wild type p53 (e.g., luciferase specific sequence). Tissues, in which the fusion is being expressed, is determined by northern blot analysis and by RT-PCR (reverse transcriptase-PCR) at different stages of development. These results are correlated with published results of p53 distribution in adult animals as well as developing animals.

Imaging of p53 Induction in the Skin of “Knock-in” Mice

The skin of knock-in mice whose p53 locus has been altered to express the p53 Luc fusion is UV-irradiated and the effect of irradiation on the induction of p53 (transiently) is examined by bioluminescence imaging as well as by immunocytochemistry. This study determines the utility of the knock-in mouse and its utility as a reporter for DNA damage. Animals have their backs shaved with electric clippers and are UV-irradiated at two doses and bioluminescence imaging is performed at two time points. Determination of the appropriate dosage and time points are performed as previously discussed. Also as described above, skin specimens are used to identify the presence of cells wherein the levels of the p53-Luc fusion have increased (using a p53 and/or a luciferase antibody). These two modalities are correlated to determine the sensitivity, validity and reproducibility of using the “knock-in” mouse as a model for non-invasive reporting of DNA damage.

### Example 4

### Luciferase as a Sensitive and Valid Reporter for Non-invasive Imaging

Current assessment of orthotopic tumor models in animals utilizes survival as the primary therapeutic endpoint. In vivo bioluminescence imaging (BLS) is a sensitive imaging modality that is rapid and accessible, and comprises an ideal tool for evaluating antineoplastic therapies. Using human tumor cell lines constitutively expressing luciferase, the kinetics of tumor growth and response to therapy have been assessed in intraperitoneal, subcutaneous and intravascular cancer models. However, use of this approach for evaluating othotopic tumor models has not been demonstrated. Therefore, the ability of BLI to noninvasively quantitate the growth and therapeutic-induced cell kill of orthotopic rat brain tumors derived from 9L gliosarcoma cells genetically engineered to stably express firefly luciferase (9L^{Luc}) was investigated.

Intracerebral tumor burden was monitored over time by quantifying photon emission and tumor volume using a cryogenically-cooled CCD camera and magnetic resonance imaging (MRI), respectively. Excellent correlation (r=0.91) between detected photons and tumor volume was found. A quantitative comparison of tumor cell kill determined from serial MRI volume measurements and BLI photon counts following 1,3-bis(2-chloroethyl)-1 nitrosourea (BCNU) treatment revealed that both imaging modalities yielded statistically similar cell kill values (p=0.951). These results provide direct validation of BLI imaging as a powerful and quantitative tool for the assessment of antineoplastic therapies in living animals.

FIGS. 4A-4J show the kinetics of intracranial glioma growth in a representative animal. 9L^{Luc} cells were implanted intracerebrally at 16 days prior to sham treatment with ethanol vehicle. Tumor progression was monitored with MRI (FIGS. 4A-4D) and BLI (FIGS. 4E-4H). The days, post sham treatment, on which the images were obtained are indicated at the top of the diagrams. The MRI images are T₂-weighted and are of a representative slice from the multi-slice dataset. The scale to the right of the BL images (FIG. 4I) describes the color map for the luminescent signal. Correlation of tumor volume with in vivo photon emission is shown where tumor volume was measured from T₂-weighted NM images and plotted against total measured photon counts (FIG. 4J). The relationship between the two measurements is defined by regression analysis (r=0.91).

FIGS. 5A-5O show a temporal analysis of the response of a 9L^{Luc} tumor to BCNU chemotherapy. Tumor cells were implanted 16 days prior to treatment. Tumor volume was monitored with T₂-weighted MRI (FIGS. 5A-5F) and infra-tumoral luciferase activity was monitored with BLI (FIGS. 5G-5L). The days post BCNIJ therapy on which the images were obtained are indicated at the top of the images. The scale to the right of the BLI images (FIG. 5M) describes the color map for the photon count. Quantitative analysis of tumor progression and response to BCNU chemotherapy is shown by the graph FIG. 50. Tumor volumes (●) and total tumor photon emission (▪) obtained by T₂-wieghted NM and BLI, respectively, are plotted versus days post BCNU treatment. The dashed lines are the regression fits of the exponential tumor repopulation following therapy. The solid vertical lines denote the apparent tumor-volume and photon-production losses elicited by BCNU on the day of treatment from which log cell kill values were calculated as previously described. Comparison of log cell kill values determined from MRI and BLI measurements are shown in the bar graph FIG. 5N. Log cell kill elicited by BCNU chemotherapy was calculated using MRI (1.78±0.36) and BLI (1.84±0.73). Data are represented as mean ±SEM for each animal (n=5). There was no significant difference between the log kills calculated using the MRI and BLI data (p=0.951).

FIG. 6 depicts one strategy for imaging apoptosis. In this example, an estrogen regulatory domain results in silencing of luciferase due to sequestration. Activation of caspase-3 during apoptosis results in cleavage at the DEVD site, thus, releasing luciferase from the silencing effects of the estrogen regulatory domain. The free luciferase can, in the presence of luciferin, generate bioluminescense which can be imaged in vitro or in vivo using a Xenogen camera system.

FIG. 7 depicts a western blot showing induction of apoptosis. In D-54 cells, a caspase-3 reporter construct analogous to the one described in FIG. 6 except with ER domains as well as the caspase-3 cleavage sequence (DEVD) were present on the amino and carboxy termini (ER-Luc-ER) was used in the above studies. This molecule was shown to have the best signal to noise ratio. D-54 cells expressing this molecule were treated with TRAIL (TNF-related apoptosis inducing ligand) at various concentrations for 3 hrs. As seen in the top panel using a luciferase specific antibody, the ER-Luc-ER molecule (120 kD) is cleaved to a 90 kD molecule (ER-Luc) and subsequently to Luc (60 kD) when aImptosis is occurring. This also correlated with the conversion of inactive zymogen caspase (32 kD) to active caspase-3 (17 kD and 13 kD).

FIGS. 8A and 8B depict non-invasive imaging of apoptosis. D-54 derived stable cell line expressing a caspase-3 reporter molecule similar to that described in FIG. 6 was implanted s.q. into nude mice. When the tumors reached a palpatable size they were treated with vector only (PBS, Panel A) or with 50 ug of TRAIL intratumoraly (panel B). Bioluminescent activity within the tumor was then measured after injection of luciferin using a IVIS imaging system. As seen in the image, there was a large (2-3×) increase in bioluminescence when apoptosis was induced using TRAIL compared to the vector control.

A number of embodiments of the invention have been described. Nevertheless, it will be understood that various modifications may be made without departing from the spirit and scope of the invention. For example, in addition to the construct provided in the above examples, the invention includes other proteins of the ubiquitin-proteosome pathway as well as other reporter molecules and non-invasive imaging means. Accordingly, other embodiments are within the scope of the following claims. The contents of the patents and publications mentioned herein are incorporated by reference in their entirety.

We claim:

1. A method for in vivo monitoring of levels of a chimeric polypeptide comprising the following steps:
(a) providing a cell, a tissue, an organ or a whole body comprising a chimeric nucleic acid or a chimeric polypeptide, wherein the chimeric nucleic acid encodes the chimeric polypeptide and the chimeric polypeptide comprises a first domain comprising a bioluminescent or a chemiluminescent polypeptide, and a second domain comprising a polypeptide of interest;
(b) expressing the chimeric polypeptide in the cell, tissue, organ or whole body or contacting the cell, tissue, organ or whole body with the chimeric polypeptide; and
(c) imaging the cell, tissue, organ or whole body to monitor the level of the chimeric polypeptide of interest in the cell, tissue, organ or whole body, wherein the image is generated by computer assisted tomography (CAT), magnetic resonance spectroscopy (MRS), magnetic resonance imaging (MRI), positron emission tomography (PET), single-photon emission computed tomography (SPECT), bioluminescence imaging (BLS) or equivalents.

2. The method of claim 1, wherein the polypeptide of interest is capable of being ubiquitinated.

3. The method of claim 2, wherein the ubiquitinated polypeptide is degraded by the ubiquitin-proteasome pathway.

4. The method of claim 1, wherein the polypeptide of interest comprises a tumor suppressor protein.

5. The method of claim 4, wherein the tumor suppressor protein comprises a p53 polypeptide or a p73 polypeptide.

6. The method of claim 1, further comprising identifying a putative modulator of the amount of the polypeptide in a cell, tissue, organ or whole body by administering a test compound or a test event to the cell, tissue, organ or whole body before, during and/or after step (b) and monitoring a change in the level of the polypeptide of interest over at least two time points to measure a change in the amount of the bioluminescent or chemiluminescent chimeric polypeptide in the cell, tissue, organ or whole body, thereby identifying the test compound or test event as a modulator of chimeric polypeptide levels.

7. A method for in vivo identification of a DNA damaging stimulus or a DNA damaging compound comprising the following steps:
(a) providing a cell, a tissue, an organ or a whole body comprising a chimeric nucleic acid or a chimeric polypeptide, wherein the chimeric nucleic acid encodes the chimeric polypeptide and the chimeric polypeptide comprises a first domain comprising a bioluminescent or a chemiluminescent polypeptide, and a second domain comprising a polypeptide that is upregulated or downregulated in response to DNA damage;
(b) providing a test compound or a test stimulus;
(c) expressing the chimeric polypeptide in the cell, tissue, organ or whole body or contacting the cell, tissue, organ or whole body with the chimeric polypeptide;
(d) administering the compound or stimulus to the cell, tissue, organ or whole body, wherein the administration can be before, during and/or after step (c); and
(e) imaging the cell, tissue, organ or whole body to monitor the level of the polypeptide in the cell, tissue, organ or whole body, wherein a change in the level of the polypeptide in response to administration of the compound identifies the test compound as a DNA damaging agent;
wherein the image is generated by computer assisted tomography (CAT), magnetic resonance spectroscopy (MRS), magnetic resonance imaging (MRI), positron emission tomography (PET), single-photon emission computed tomography (SPECT), bioluminescence imaging (BLS) or equivalents.

8. The method of claim 7, wherein the polypeptide upregulated or downregulated in response to DNA damage comprises a tumor suppressor protein.

9. The method of claim 8, wherein the tumor suppressor protein comprises a p53 polypeptide or a p73 polypeptide.

10. The method of claim 7, wherein the stimulus comprises administration of a chemical or a radiation to the cell, tissue, organ or whole body.

11. A method for in vivo monitoring ubiquitin-proteasome pathway activity, comprising the following steps:
(a) providing a cell, a tissue, an organ or a whole body comprising a chimeric nucleic acid or a chimeric polypeptide, wherein the chimeric nucleic acid encodes the chimeric polypeptide and the chimeric polypeptide comprises a first domain comprising a bioluminescent or a chemiluminescent polypeptide, and a second domain comprising a polypeptide whose cellular levels are regulated by ubiquitination;
(b) expressing the chimeric polypeptide in the cell, tissue, organ or whole body or contacting the cell, tissue, organ or whole body with the chimeric polypeptide; and
(c) imaging the cell, tissue, organ or whole body to monitor the level of the polypeptide in the cell, tissue, organ or whole body, wherein a change in the level of the polypeptide is an indication of ubiquitin-proteasome pathway activity, wherein the image is generated by computer assisted tomography (CAT), magnetic resonance spectroscopy (MRS), magnetic resonance imaging (MRI), positron emission tomography (PET), single-photon emission computed tomography (SPECT), bioluminescence imaging (BLI), or equivalents.
